(19) United States
(12) Patent Application Publication
(10) Pub. No.: US 2014/0302493 A1
(43) Pub. Date: Oct. 9, 2014

(54) METHOD AND QUALITY CONTROL MOLECULAR BASED MOUSE EMBRYO ASSAY FOR USE WITH IN VITRO FERTILIZATION TECHNOLOGY

(71) Applicant: Irvine Scientific Sales Company, Inc., Santa Ana, CA (US)

(72) Inventors: Hsiao-Tzu Ni, Irvine, CA (US); Samira Es-Slami, Santa Ana, CA (US); Rebecca Susan Gilbert, Lake Forest, CA (US)

(21) Appl. No.: 14/214,298

(22) Filed: Mar. 14, 2014

Related U.S. Application Data
(60) Provisional application No. 61/783,557, filed on Mar. 14, 2013.

(51) Int. Cl. C12Q 1/68

(52) U.S. Cl. CPC C12Q 1/68
USPC 435/6.1; 435/325

(57) ABSTRACT

A method for qualitatively assessing products used in in vitro fertilization is provided. Also disclosed is an improved quality control assay for use in clinical Assisted Reproductive Technologies (ART).

### BACKGROUND OF THE INVENTION

1. Field of the Invention

The present invention relates to a method for assessing products used in in vitro fertilization. Also disclosed is a quality control assay for use in clinical Assisted Reproductive Technologies (ART).

2. Description of the Related Art

The in vitro fertilization (IVF) laboratory plays a fundamental role in the delivery of treatment to infertile couples. Ensuring proper Quality Control (QC) in the IVF laboratory is critical to the success of any IVF program as the environment of the laboratory can alter the quality of the embryos produced. An optimal culture medium and a stable environment are necessary for the successful development of human embryos in vitro. The ultimate role of the embryology laboratory is to maintain the inherent viability of the gametes and embryos in an environment outside the female reproductive tract. The dynamic nature of pre-implantation embryo development is unique because, unlike somatic cell culture, embryos are constantly changing, both in morphology and function, every day (Leese 1991; Bavister 1995).

It is during this time that the pre-implantation embryo changes rapidly, in just a matter of days, from a metabolically quiescent, undifferentiated single cell under genetic control from maternal transcripts into a dynamic, multi-celled embryo that has developed homeostatic mechanisms and its own functioning genome (Leese 1991; Lane 2001; Gardner et al. 2005). The embryo changes from a pyruvate-based metabolism solely dependent on mitochondrial oxidative phosphorylation for energy production and resembles a unicellular organism lacking many key regulatory functions for pH and osmotic control. After compaction at the eight- to 16-cell stage (dependent on species), there is a change in metabolic control to a highly glycolytic metabolism. Concomitantly, there is also a marked transition in the functional complexity of other cellular mechanism as the embryo's physiology becomes more like that of a somatic cell. It is the initially crude nature of homeostatic regulation in the early embryo and its subsequent development through later stages of pre-implantation development that pose significant challenges in the laboratory. Maintenance of a favorable in vitro environment is essential for maximizing viability and ongoing development.

Perturbations to the environment surrounding the embryo during development in culture, relative to “normal” conditions encountered in the reproductive tract, result in reduced embryo viability and impaired development. However, it is often difficult to assess the impact of suboptimal environment using morphology as a marker. In certain instances, embryos can develop to apparently morphologically normal blastocysts but at the cellular level, these embryos can be compromised and have a reduced capacity to implant and produce a successful term pregnancy. The environment that an embryo is exposed to during collection and culture can significantly alter its developmental potential and cellular regulation.

The mouse embryo assay (MEA) has been the gold standard to examine the applicability of culture media and environment without involving human materials. The basic techniques and protocols employed for performing the MEA are set forth in In Vitro Fertilization And Embryo Transfer: A Manual of Basic Techniques (Don P. Wolf, Editor), 1988. pages 57-75; the contents of which are hereby incorporated by reference in their entirety. Briefly, the assay involves superovulation of female mice with PMSG (pregnant mare serum gonadotropin) and hCG (human chorionic gonadotropin). The mice are placed with males at the time of hCG injection and killed 24 hours following hCG to obtain on-cell embryos and 36 hours after injection to obtain two-cell embryos. One-cell embryos are selected for use if they have two polar bodies visible, and two cell embryos are selected for use if they look morphologically normal.

Specifically, the MEA is used for toxicity and functionality testing of reproductive media, labware, or any device coming into contact with gametes and/or embryos. The rationale for requiring information on this test as a special control for class II assisted reproduction devices is that it is a good surrogate indicator of potential toxicity of materials used in assisted reproduction devices to gametes and/or embryos. The FDA has recognized that the MEA is currently the most appropriate test for embryo toxicity. Briefly, both one-cell and two-cell assays are used, and these are identical except that one-cell embryos are flushed from the mouse oviduct earlier than two-cell embryos. If the MEA is performed, whether a one-cell or two-cell MEA is used, the bioassay should represent, as closely as possible, the corresponding procedures used for which the device is used for human IVF, such as the acquisition, maintenance, culture, transfer (relocation) and cryopreservation of embryos. Typically, embryo morphology is assessed and blastocyst formation is determined after 96 hours of culture. If more than 80% of the zygotes have reached the blastocyst stage, the medium, or equipment tested, are considered suitable for clinical use.

In addition to detecting embryo toxicity, the MEA is capable of detecting suboptimal raw materials, media, and contact materials associated with IVF and ART. However, there are a number of limitations of this assay which are often overlooked. For example, the assay can only detect conditions which are grossly and harshly embryo toxic. The MEA cannot detect or differentiate growth promoting or inhibiting factors at a very early stage in development.

There is a need for objective, sensitive, and reproducible methods and assays for testing materials used in human IVF for embryo toxicity as well as growth promoting and inhibiting factors.

### SUMMARY OF THE INVENTION

Embodiments described herein are directed to systems and methods for providing a molecular based mouse embryo assay for use as a quality control in in vitro fertilization arenas and/or Assisted Reproductive Technologies (ART), and more specifically to an improved assay for assessing embryonic development from one-cell or two-cell to blastocyst stage.

From this description, in conjunction with other items, the advantages of the invention will become clear and apparent more so based upon the hereinafter descriptions and claims, which are supported by drawings with numbers relating to parts, wherein are described in the following sections containing the relating numbers.

In one aspect, a quality control method for assessing products used for human in vitro fertilization; or Assisted Reproductive Technologies (ART) is provided. The method includes providing a transgenic embryo (at least one-cell) and culturing the embryo in vitro for a specified period of time. The method further includes evaluating the embryonic development from one-cell or two-cell to the blastocyst stage and beyond. Acceptability or failure of the tested items is determined based upon qualitative and quantitative analyses of the embryo development. Optionally, the one-cell embryo includes at least one fluorescent protein transgene operably linked to at least one embryonic development/pluripotency regulator.

The transgene may include a reporter gene encoding a selected fluorescent protein such as green fluorescent protein (GFP), red fluorescent protein, Cyan Florescent protein, Orange Fluorescent protein or yellow fluorescent protein.

In another aspect, the quality control method and assay are designed to evaluate test items used in in vitro fertilization environments and/or Assisted Reproductive Technologies (ART). The test items may include gamete and embryo culture media, gamete and embryo handling/processing media (to include washing and separation media), transport media, enzymes for denuding oocytes, gradient for sperm separation, freezing/vitrification media, thawing/warming media, pipette and embryo handling devices, lab-ware used in the process of human in vitro fertilization including but not limited to Petri dishes, centrifuge tubes, cryopreservation and Cryo-storage devices, and any solutions, reagents or devices involved with in vitro ART related procedures.

In another aspect, evaluation of embryonic development is accomplished by general embryo morphology related to the developmental stages of the embryos and the location/quantity/quality of fluorescence. Preferably, embryo is derived from mammalians and can include murine, porcine, equine, bovine, ovine and non-human primate embryo.

In still another aspect, the operably linked embryonic pluripotency regulator may include without limitation Oct4, Sox2, Nanog, CDX2 and Rexl as well as their upstream mediators and downstream effectors that play a role in ensuring normal embryo development.

Embryo development may be assessed at all stages including 1 and 2-cell-stages, 4-cell stage, 8-cells stage, morula stage, blastocyst stage and gastrulation stages.

A quality control assay for use in clinical ART to evaluate products used in the process of handling and preserving human gametes and producing, culturing and preserving human embryos is likewise provided. The assay advantageously includes a transgenic one-cell embryo harvested from a transgenic mammal, wherein the embryo comprises at least one reporter gene operably linked to at least one embryonic pluripotency marker; and instructions for evaluating ART products and IVF culture conditions. The instructions can include incubating a transgenic one-cell embryo under certain culture conditions and evaluating embryo development based upon morphology from the one and two-cell to blastulation and gastrulation stages.

Optionally, the reporter gene encodes a protein such as fluorescent proteins like Green Fluorescent Protein, Cyan Fluorescent Protein, Orange Fluorescent Protein, Yellow Fluorescent Protein.

In another aspect, the assay includes embryonic pluripotency regulators, their upstream mediators and downstream effectors that play a role in ensuring normal embryo development. The test items/growth conditions may be evaluated based on embryo growth, development and quality based upon assessment of embryo morphology and qualitative/quantitative assessment of fluorescence. The acceptable threshold for optimal embryo growth and development is based on individual set criteria depending on test items and expected development under normal/control conditions. In the event that the test items do not meet the established acceptance criteria compared to a normal control, they would be considered suboptimal or embryotoxic.

In still another aspect of the invention, an enhanced embryo assay (EEA) for use in quality control of clinical human ART/IVF is described. The assay may include a transgenic one-cell embryo. The embryo can include at least one reporter gene operably linked to at least one gene associated with embryonic development. Preferably, the embryo expresses a transgenic/reporter gene differentially under optimal and sub-optimal culture conditions. In another aspect, the culture conditions are embryo-toxic. Also provided is a test item such as, for example, embryo culture media, gamete handling media, enzymes for denuding oocytes, gradient for sperm separation, freezing media, thawing media, pipettes and embryo handling devices, lab-ware used in the process of human in vitro fertilization including but not limited to Petri dishes, centrifuge tubes, cryopreservation and cryostorage devices.

The invention disclosed herein further includes a method for enhancing the sensitivity of embryo assay using embryo development to the blastocyst stage. The method includes providing a transgenic embryo comprising at least one reporter gene operably linked to at least one embryonic pluripotency marker; incubating the transgenic embryo under culture conditions/test items; and evaluating embryo development morphologically and via the expression of said embryonic marker from one-cell to blastocyst and gastrulation stages.

Optionally, the method for enhancing the sensitivity of an embryo assay further includes evaluating expression of the embryonic marker at the blastocyst stage and beyond (gastrulation). The evaluation may comprise determining fluorescence of the reporter gene. The assay may detect embryo-toxicity in culture media and/or culture materials. In one aspect, the assay detects functionality of media and suitability of materials used in clinical in vitro fertilization environments.

A modified, transgenic embryo, comprising at least one transgene operably linked to at least one embryonic pluripotency regulator is disclosed. The embryonic pluripotency regulators include these regulators' genes as well as their upstream mediators and downstream effectors that play a role in ensuring normal embryo development. Advantageously, the transgene is a reporter gene. The reporter gene may be a fluorescent or luminescent protein such as green fluorescent protein, red fluorescent protein, cyan fluorescent protein, orange fluorescent protein, or yellow fluorescent protein.

### BRIEF DESCRIPTION OF THE DRAWINGS

FIG. 1 is a color photograph of mouse embryo incubated from the 2-cell to blastocyst stage under optimal and sub-optimal IVF growth conditions. The mouse embryo comprises the OCT4 embryonic pluripotency regulator linked to a fluorescent tag.

FIG. 2A is a color photograph of a molecular-based mouse embryo assay under optimal growth conditions at the blastocyst stage under fluorescent microscopy.

FIG. 2B is a color photograph of a molecular-based mouse embryo assay under sub-optimal IVF growth conditions. The embryo is a blastocyst photographed under fluorescent microscopy.

FIG. 3A is a color photograph of a SOX-2 study in optimal growth conditions.

FIG. 3B is a color photograph of a mouse embryo with the SOX-2 pluripotency regulator at the blastocyst stage in sub-optimal growth conditions.

FIG. 3C is a fluoro microscopic photograph of mouse embryos at the blastocyst stage of development and having the SOX-2 pluripotency marker linked to a fluorescent tag.

### DETAILED DESCRIPTION OF THE PREFERRED EMBODIMENT

After reading this description it will become apparent to one skilled in the art how to implement the invention in various alternative embodiments and alternative applications. However, all the various embodiments of the present invention will not be described herein. It is understood that the embodiments presented here are presented by way of an example only, and not limitation. As such, this detailed description of various alternative embodiments should not be construed to limit the scope or breadth of the present invention as set forth below.

A quality control method for assessing the culture conditions for in vitro fertilization is disclosed. Fertilized embryos harvested from reporter-transgenic mice are used to detect detrimental or sub-optimal culture conditions. Effectively, the transgenic embryos provide a more sensitive and functionally relevant qualitative quality control (QC) assay for testing and qualifying devices for use in clinical in vitro fertilization laboratories.

As will be described in greater detail below, the method includes providing a transgenic at least one-cell blastocyst; culturing the blastocyst under in vitro or Assisted Reproductive Technology (“ART”) culture conditions, and evaluating blastocyst differentiation at the two-cell blastocyst stage to determine the acceptability of the culture conditions.

“Assisted Reproductive Technology” or ART, as used herein, includes all fertility treatments in which both female gametes (eggs or oocytes) and male gametes (sperm) are handled. In Vitro Fertilization (IVF) is one of several assisted reproductive techniques used to assist infertile couples in conceiving a child. IVF refers to the procedure by which eggs are removed from the female's ovary and fertilized with sperm in a laboratory procedure. The fertilized egg (embryo) can be cryopreserved for future use or transferred to the uterus. As used herein, “blastocyst” refers to a structure in early embryonic development consisting of a ball of cells with surrounding wall (trophectoderm) which will form the placenta and a fluid filled cavity (blastocoels) which will form the amniotic sac and an internal cluster of cells called the inner cell mass from which the fetus arises.

Quality control assays and methods of performing quality control assays as described in detail below include a mammalian transgenic embryo (at least one-cell). In preferred embodiments, the embryo is at one or two-cell stage. The mammalian embryo can be obtained from bovine, ovine, porcine, murine, canine, equine, simian, or human origin. Preferably, the mammalian embryo is porcine, equine, or bovine. More commonly, the embryo is murine derived.

The term “transgenic” means of or pertaining to a segment of DNA that has been incorporated into a host genome or is capable of autonomous replication in a host cell and is capable of causing expression of one or more cellular products. Exemplary transgenes will provide the host cell, or animals developed therefrom, with a novel phenotype relative to the corresponding non-transformed cell or animal. “Transgenic animal” means a non-human animal, usually a mammal, having a non-endogenous nucleic acid sequence present as an extrachromosomal element in a portion of its cells or stably integrated into its germ line DNA.

Transgenesis is used to create transgenic mammals such as mice with reporter genes linked to a gene of interest. Methods in molecular genetics and genetic engineering are described generally in the current editions of Molecular Cloning: A Laboratory Manual, (Sambrook et al.); Oligonucleotide Synthesis (M. J. Gait, ed.); Animal Cell Culture (R. I. Freshney, ed.); Gene Transfer Vectors for Mammalian Cells (Miller & Calos, eds.); Current Protocols in Molecular Biology and Short Protocols in Molecular Biology, 3.sup.rd Edition (F. M. Ausubel et al., eds.); and Recombinant DNA Methodology (R. Wu ed., Academic Press). Thus, transgenic technology is well established. See, Transgenic Mouse: Methods and Protocols (M. Hofker and J. Deursen, Eds.) in Methods in Molecular Biology (Vol. 209) (the contents of which are hereby incorporated by reference in their entirety).

The transgenic mammal includes a reporter gene linked to a viability marker or pluripotency gene of interest. Reporter genes include, for example, fluorescent or luminescent protein such as luciferase, green fluorescent protein, or red fluorescent protein. Fluorescent proteins can include, without limitation, blue/UV proteins such as TagBFP, mTagBFP2, azurite, EBFP2, mKalamal, Sirius, sapphire, and T-sapphire. Fluorescent proteins can also include cyan proteins such as ECFP, cerulean, SCFP3A, mTurquoise, mTurquoise2, monomeric Midoriishi-Cyan, TagCFP, and mTFP1. In a preferred embodiment, the fluorescent protein is a green protein such as EGFP, Emerald, Superfolder GFP, Monomeric Azami Green, TagGFP2, mUKG, mWasabi, or Clover. Yellow fluorescent proteins including EYFP, Citrine, Venus, SYFP2, ZsYellow1, and TagYFP are likewise contemplated for use as a reporter gene. Orange proteins for use as reporter genes can include Monomeric Kusabira-Orange, mKOₖ, mKO2, mOrange, and mOrange2. Red proteins such as HcRed1, mRaspberry, mCherry, mStrawberry, mTangerine, tdTomato, TagRFP, mApple, mRuby, and mRuby2. Far-red proteins include, without limitation, mPlum, HcRed-Tandem, mKate2, mNeptune, and NirFP. The embryos of transgenic mice express the reporter proteins every time the markers of interest are expressed.

As used herein, viability markers include embryonic development/pluripotency markers. Generally, these markers include embryonic stem cell associated transcript genes. Pluripotent stem cell markers, as used herein, are expressed at a predictable level and location at a predictable time of embryonic development. Pluripotent stem cell (PS)-specific markers include, but are not limited to, the family of octamer transcription factors, i.e. Oct4; the family of Sox genes, i.e. Sox 1, Sox2, Sox3, Sox 15, and Sox 18; the family of Klf genes such as Klf4 and Klf5; the family of Nanog genes, i.e. NANOG, as well as their upstream mediators and downstream effectors. Other markers can also include, without limitation, the TGF-beta superfamily and their receptors, i.e. Activ RIB/ALK-4, GDF-3 and Lefty, the cryptic protein family, i.e. Cripto-1, the integrin family, i.e. integrin alpha 6 (CD49f) and integrin beta 1 (CD29), the Podocalyxin family, i.e. PODX-1, the FGF family, i.e. FGF4 and FGF-5, the Forkhead box transcription factor family, i.e. FoxD3, the T-box family of transcription factor, i.e. TBX3 and TBX5, the family of developmental pluripotency associated molecules, i.e. Dppa2, Dppa3/Stella, Dppa4 and Dppa5/ESG1, the LRR family, i.e. 5T4, the cadherin family, i.e. E-Cadherin, the connexin family of transmembrane proteins, i.e. Connexin-43 and Connexin-45, the F-box family of “other” category, i.e. FBOXO15, the family of chemokine/chemokine receptors i.e. CCR4 and CXCR4, the ATP-binding Casstet Transporters, i.e. ABCG2. Additional common known markers involved in OCT4 and/or SOX2-mediated stemness maintenance are Utf1, TERT, Zscan4, CD9, CD15/Lewis X, CD25, CD30/TNFRSF8, CD90/Thyl, Alkaline Phosphatase/ALPL, alpha HCG, HCG, DNMT3B, GBX2, GCNF/NR6A1, Gi24/Dies1/VISTA, LIN-28A, LIN-28B, LIN-41, c-Myc, Rex-1/ZFP42, sFRP-2, Smad2, Smad2/3, SPARC, STATS, SUZ12, TOBX2, TEX19/19.1, THAP11, and TROP-2.

Qualitative analysis of embryo development is accomplished by analyzing the developing embryo via light microscopy which may include UV light to visualize fluorescent protein expression. The blastocyst differentiation can be evaluated via confocal microscopy. In a preferred embodiment, acceptability of culture conditions is based upon the qualitative analysis of embryo development via fluorescence microscopy. In a particularly preferred embodiment, embryonic development is observed via an embryo scope, wherein a picture of developing embryos can be taken approximately every 10 minutes and a time-lapse video can be generated to track all stage of embryo development. As illustrated in the Figures (as will be described in greater detail with reference to the Examples), embryo development is determined both in terms of chronology (stage reached for a specific culture duration) and embryo quality both morphologically and functionally by assessing the location and quantity/intensity of the fluorescence. Expression of single markers in a test cell will provide evidence of undifferentiated or differentiated phenotype, according to the expression pattern. Expression of genes that are down-regulated and/or lack of expression of genes that are up-regulated upon development/differentiation.

The culture of gametes and embryos is an integral part of any reproductive research laboratory, as is the use of plastic-ware and other consumables, such as gloves, media, chemicals, and oil. In the IVF setting, the quality control of all consumable and plastic ware is important for maintaining an optimal environment for embryo culture, thus ensuring normal embryo physiology and subsequent pregnancy rates. Thus, in one embodiment, a method of evaluating embryo toxicity of IVF consumables is provided. As used herein, IVF consumables include, without limitation, plasticware, tubing, pipettors, etc. or any material that comes into contact with human eggs or embryos is provided. Plasticware can include assisted reproduction needles, laboratory gloves, assisted reproduction catheters, and assisted reproduction microtools such as pipettes or other devices used in the laboratory to denude, micromanipulate, hold, or transfer embryos. IVF consumables further include assisted reproduction labware, including without limitation, syringes, IVF tissue culture dishes, IVF tissue culture plates, pipette tips, dishes, plates, and other vessels that come into physical contact with gametes, embryos, or tissue culture media. As used herein, IVF consumables can include assisted reproduction water and water purification systems intended to generate high quality sterile, pyrogen-free water for reconstitution of media used for aspiration, incubation, transfer or storage of embryos for IVF or other assisted reproduction procedures as well as for use as the final rinse for labware or other assisted reproduction devices which will contact the embryos.

A method and assay for evaluating embryo toxicity associated with culture media with a higher level of sensitivity than standard MEA assays are likewise provided. The method includes providing an assay comprising a transgenic embryo having a reporter gene operably linked to a pluripotency marker. Also provided is a control medium which promotes normal embryo development. As used herein, culture media includes, without limitation, reproductive media and supplements used for assisted reproduction procedures. Media include liquid and powder versions of various substances which come in direct physical contact with embryos (e.g. water, acid solutions used to treat gametes or embryos, rinsing solutions, reagents, sperm separation media, or oil used to cover the media) for the purposes of preparation, maintenance, transfer or storage. Supplements, as used herein, include specific reagents added to media to enhance specific properties of the media such as proteins, sera, antibiotics, or the like.

Test culture media is compared to control media by assessing embryo development at 2-cell-stages, 4-cell stage, 8-cells stage, morula stage, blastocyst stage and gastrulation stages. More particularly, the transgenic embryos are analyzed microscopically to assess differentiation at the blastocyst stage of development. For example, in the case of transgenic murine embryos, the embryos are assessed at approximately 96 hours after fertilization. For other mammalian embryos, the duration of time from fertilization to blastocyst development can vary depending upon the source of the embryos. For example, blastocyst development for human embryos typically occurs at Day 5. Embryonic viability is assessed based upon scoring embryo morphology and qualitative/quantitative assessment of fluorescence. The acceptable threshold for optimal embryo growth is based on individual set criteria depending on culture conditions and test items. In each test, a control benchmark is run in parallel with the test culture medium. For example, when new medium is evaluated for use in an IVF environment, the medium is tested against a control medium which has been pre-determined to provide optimal growth conditions for embryos. New test culture medium is evaluated by assessing blastocyst development relative to the blastocyst development in the control medium. Assessment can include a qualitative comparison of the number of cultured embryos reaching the blastocyst stage in the control medium as compared to the number of embryo reaching the same stage in the test medium. Acceptable quality control permits an at least 80% blastocysts in the test medium. Additional growth parameters include the number of cells observed at the blastocyst stage in the control versus the test medium as well as the intensity and localization of fluorescence of the reporter gene in the transgenic blastocysts. As compared with the standard MEA assay, where the blastocyst may look normal, the disclosed assay provides a more enhanced sensitivity to embryo development. The reporter gene operably linked to a pluripotency/viability marker can be observed microscopically and provides a better delineation of gene expression in optimal, sub-optimal, and/or embryo toxic growth conditions. “Optimal” as used herein, refers to conditions which promote healthy, unfettered embryonic development. “Sub-optimal” conditions, by contrast, are culture conditions which allow for some cellular growth but the growth is slower and less robust than what would be predicted to be observed under optimal culture conditions. “Embryo toxicity” as used herein, refers to culture conditions which induce abnormal development or embryo death.

A quality control assay for use in clinical ART to evaluate products used in the process of handling and preserving human gametes and producing, culturing and preserving human embryos is provided. The assay includes a transgenic one-cell embryo harvested from a transgenic mammal, wherein the transgenic embryo includes at least one reporter gene operably linked to at least one embryonic pluripotency marker. The pluripotency regulator is a viability marker such as OCT-4, SOX-2, Nanog, CDX2 as well as their upstream mediators and downstream effectors that play a role in ensuring normal embryo development. The reporter gene encodes a protein. The protein can include, without limitation, Green Fluorescent Protein, Cyan Fluorescent Protein, Orange Fluorescent Protein, or Yellow Fluorescent Protein. The assay also includes instructions for evaluating ART products and IVF culture conditions. These instructions include directions relating to incubating a transgenic one-cell embryo under ART conditions and evaluating embryo development based upon morphology from the one and two-cell to later blastulation and gastrulation stages. Incubation, as used herein, describes the process by which fertilized, one or two cell embryos are cultured for approximately 72-96 hours in a defined culture media.

The suitability of a particular product for use in clinical ART is evaluated based on embryo growth, development and quality. Qualitative scoring of embryo development is based upon assessment of embryo morphology and qualitative/quantitative assessment of fluorescence. For both control and test products, the same number of one-cell or two-cell embryos are cultured in vitro. Qualitative assessment can include a comparison of embryonic development from day 0 to the blastocyst stage. One day after fertilization, for example, one would expect to observe cleavage of the embryo in both the control and embryos cultured on the test product. Two days after fertilization, in the case of murine assays, one would expect to observe an early morula stage of development under optimal conditions in both the control and test product if the test product is to be deemed acceptable for use in ART. The number of embryos that develop to the blastocyst stage is likewise quantified in both the control and test product. An assessment is made with regard to suitability of the test product for use in ART based upon the number of viable blastocysts observed as well as qualitative appearance of those blastocysts when observed microscopically. As will be seen in greater detail with reference to the Examples and Figures, morphological differences in cellular development can be observed under optimal and suboptimal culture conditions based upon location of the fluorescence (nuclear versus cytoplasmic localization, e.g.) as well as the intensity of fluorescence. As will be readily appreciated by a skilled artisan, the acceptable threshold for optimal embryo growth is based on individual set criteria depending on culture conditions and test items.

Also disclosed is an enhanced embryo assay (EEA) for use in quality control of clinical human ART/IVF. The EEA includes a transgenic one-cell embryo. The transgenic embryo includes at least one reporter gene operably linked to at least one gene associated with embryonic development; and an embryo expressing a transgenic/reporter gene differentially under optimal and sub-optimal or embryo-toxic culture conditions. The EEA further includes an ART/IVF consumable. An ART consumable can include, without limitation, embryo culture media, gamete handling media, enzymes for denuding oocytes, gradient for sperm separation, freezing media, thawing media, pipettes and embryo handling devices, lab-ware used in the process of human in vitro fertilization including but not limited to Petri dishes, centrifuge tubes, cryopreservation and cryostorage devices.

In another embodiment, a method for enhancing the sensitivity of embryo assay using embryo development to the blastocyst stage is described. The method includes providing a transgenic embryo comprising at least one reporter gene operably linked to at least one embryonic pluripotency marker, incubating the transgenic embryo under culture conditions/test items; and evaluating embryo development morphologically and via the expression of the embryonic marker from one-cell to blastocyst and gastrulation stages. The method can further include evaluating expression of the embryonic marker at the blastocyst stage and beyond (gastrulation). Evaluation of expression is measure, for example, by determining fluorescence of the reporter gene. The embryo assay can detect embryo-toxicity in culture media and/or culture materials. In another embodiment, the assay can detect functionality of media and suitability of materials used in clinical in vitro fertilization environments.

An assay for testing the effectiveness of glassware washing techniques, cleansing of surgical instruments (aspiration needle), transfer catheters and any other item that comes in contact with the human eggs, sperm or embryos is likewise described.

Also contemplated and disclosed is a modified, transgenic embryo, comprising at least one transgene operably linked to at least one embryonic pluripotency marker. The embryonic pluripotency markers and their upstream mediators and downstream effectors play a role in ensuring normal embryo development. In a preferred embodiment, the transgene is a reporter gene. The reporter gene is a fluorescent or luminescent protein selected from the group consisting of green fluorescent protein, red fluorescent protein, cyan fluorescent protein, orange fluorescent protein, yellow fluorescent protein.

### EXAMPLES

Additional embodiments are disclosed in further detail in the following examples, which are not in any way intended to limit the scope of the claims.

Several factors such as toxicity and sterility of the culture media or materials used in ART can affect development of embryos. The following examples describe assays to assess embryonic viability under optimal and sub-optimal culture conditions. Embryos in which pluripotency regulators were visualized using a fluorescent microscope are shown. The pluripotency markers used for assessing embryonic development from one or two cell stage of development include, for example, SOX-2, Oct-4, Nanog as well as their upstream mediators and downstream effectors that play a role in ensuring normal embryo development.

To test culture media, media additives, or ART consumables, the collected transgenic embryos are first incubated in 50 μL droplets of either control medium (medium which has already been determined to promote optimal blastocyst development) or test media, in each case the embryos are covered by mineral oil at 37 degrees C. in classical cell culture conditions (humidified atmosphere of 5% CO2 in air). On day 1, 2, or 3, embryos are selected for assays and transferred in the medium to be tested. The embryos are cultivated until day 5. By comparing the rates of blastocysts stages reached versus control groups, a cytotoxic or sub-optimal effect can be identified which interferes with embryo development.

### Example 1

### OCT-4

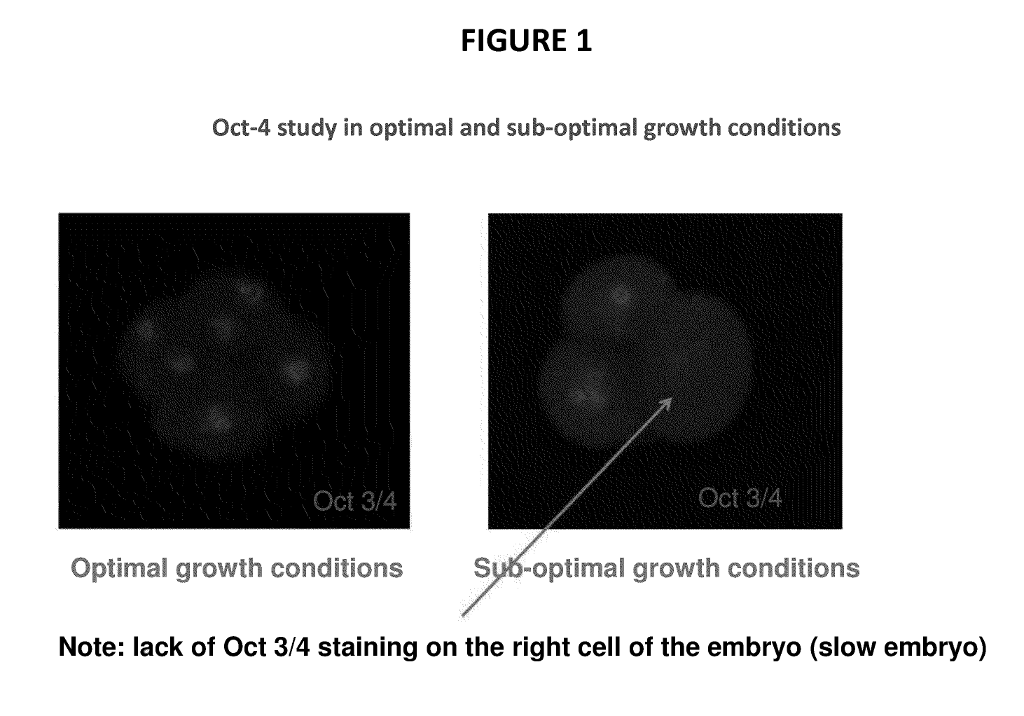

FIG. 1 is a photograph from the fluorescence microscope showing one embodiment of a molecular-based mammalian embryo assay for use in quality control. A mouse embryo with the pluripotency regulator Oct-4 visualized with red fluorescence tag. The 2-cell embryo is cultured in vitro. After approximately 48 hours, the embryos were stained and evaluated via fluororescence microscopy. As is evident from FIG. 1, the embryo on the left, which was incubated under optimal growth conditions, is well developed with uniform staining. By contrast, the blastocyst on the right was incubated in sub-optimal growth conditions. A lack of Oct-4 staining is observed on the right cell of the embryo on the right, demonstrating that the sub-optimal growth conditions result in slower embryonic development.

### Example 2

### OCT-4

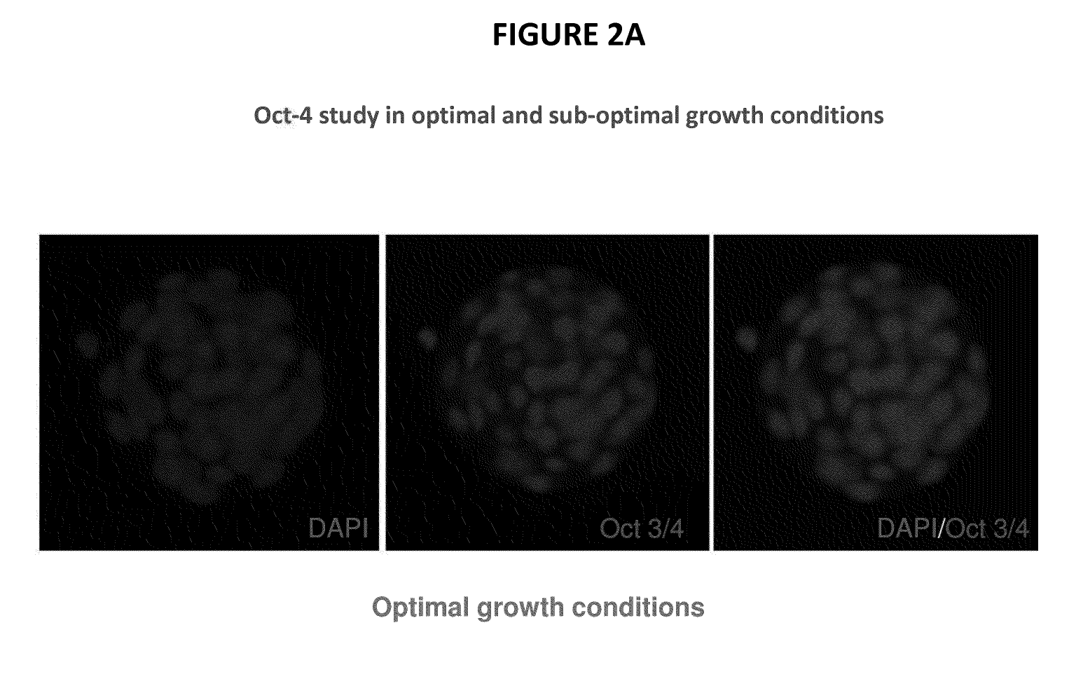

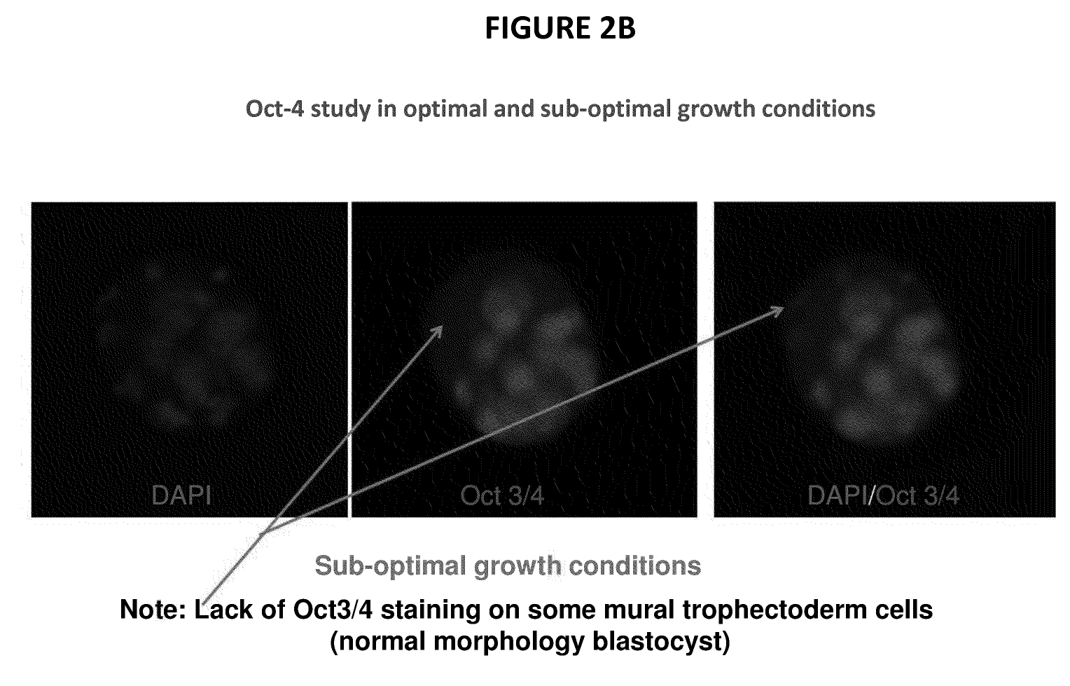

FIGS. 2A and 2B illustrate photographically a molecular-based mouse embryo assay at the blastocyst stage. The embryos were cultured in vitro for 96 hours and stained and observed via a fluorescence microscope. In FIG. 2A, under optimal growth conditions, normal embryo development is observed as demonstrated by the uniform staining. By contrast, in FIG. 2B, the embryos were incubated under sub-optimal growth conditions. A lack of Oct-4 staining on some mural trophectoderm cells is observed in the embryos as noted by the arrows on FIG. 2B. Without the use of the presently claimed technology, the sub-optimal culture conditions would not be evident or identifiable as sub-optimal when using the conventional MEA standard QC protocol because the blastocyst appears to be developing at a normal rate. However, by observing the slow growth in the center and left pictures of FIG. 2B, it is clear that blastocyst development is qualitatively less uniform and less optimal than in the embryonic development and blastocyst differentiation observed in FIG. 2A.

### Example 3

### SOX-2

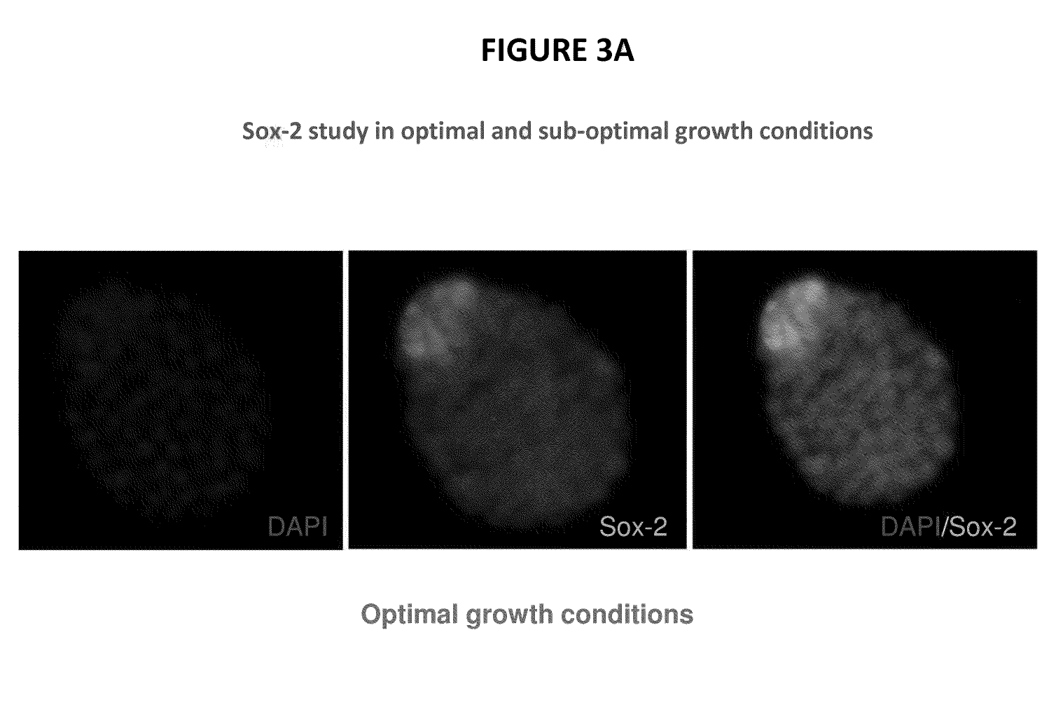

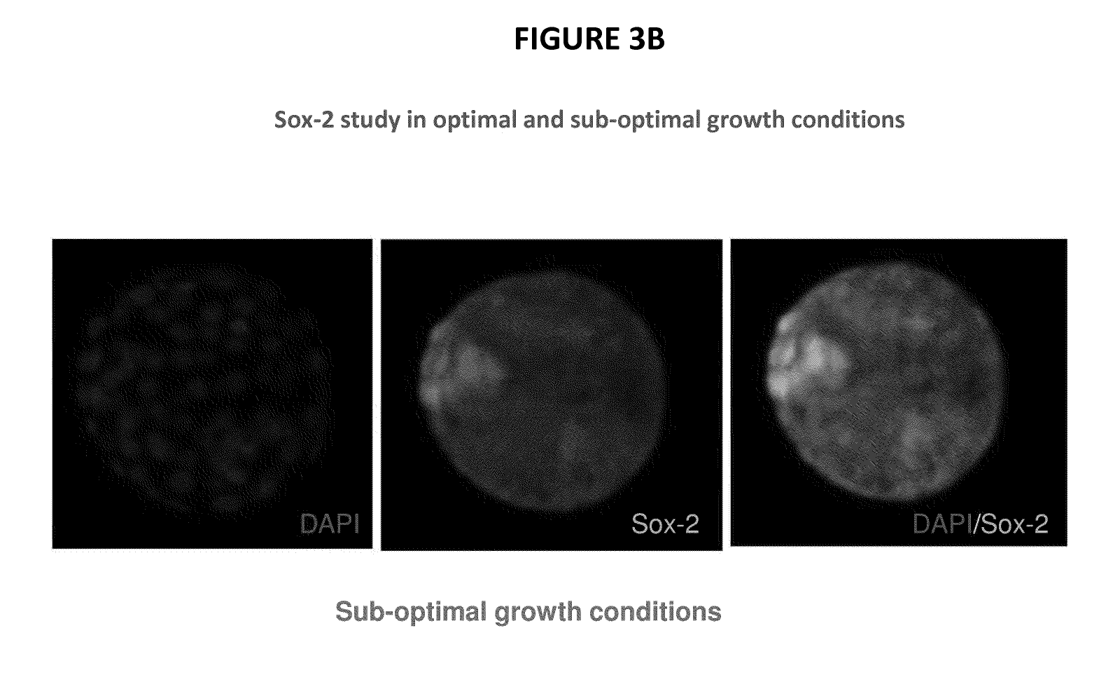

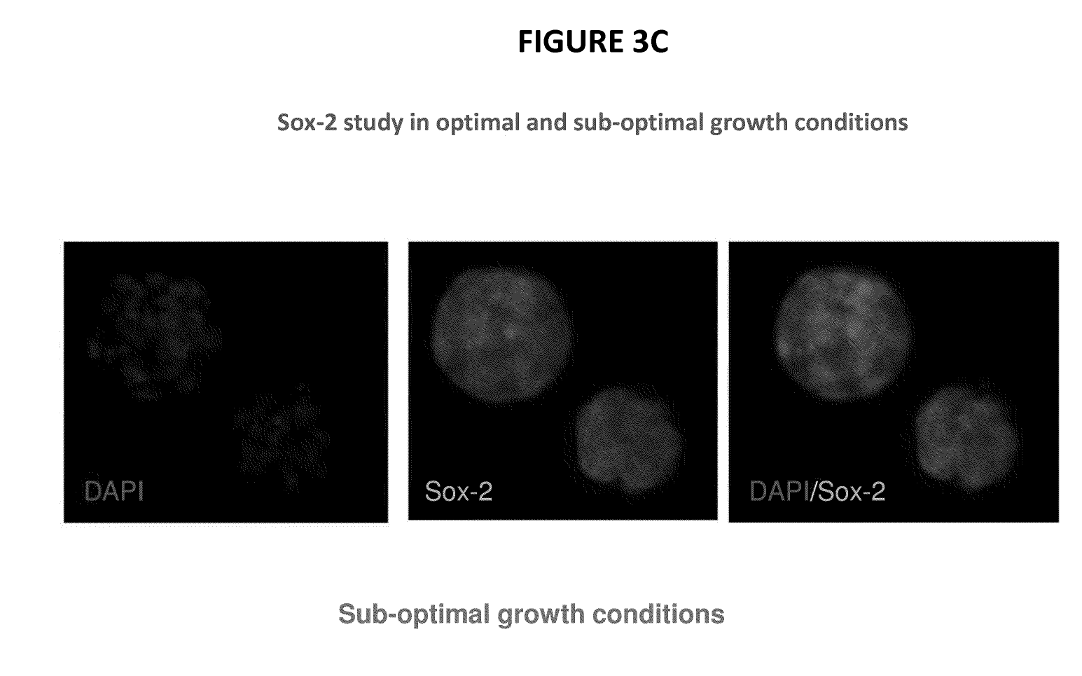

FIGS. 3A, 3B, and 3C further demonstrate the superior QC features of the claimed invention. FIGS. 3A-3C are fluorescence microscopy photographs of control murine Embryos were incubated to the blastocyst stage in vitro, stained, and observed microscopically. After 96 hours of culture, the image on the left of FIG. 3A shows an embryo that was fixed and stained with DAPI and observed microscopically to assess growth. The staining pattern observed in the embryo is uniform and evidences normal, healthy blastocyst development under optimal conditions. The center image in FIG. 3A shows the embryo having the SOX-2 viability marker stained with green fluorescence and DAPI. Notice the uniform staining as well as the well-defined differentiation of the blastocyst.

Turning to FIG. 3B, blastocysts incubated in sub-optimal growth conditions are observed. The picture of FIG. 3B shows a mouse embryo incubated under sub-optimal culture conditions. After 96 hours, the embryo is fixed and stained with DAPI and observed microscopically to assess growth. The picture appears to demonstrate normal growth and development despite the sub-optimal culture conditions.

In FIG. 3C, shows 2 embryos with poor growth in suboptimal culture conditions.

It will be understood by those of skill in the art that numerous and various modifications can be made without departing from the spirit of the present disclosure. Therefore, it should be clearly understood that the forms disclosed herein are illustrative only and are not intended to limit the scope of the present disclosure.

What is claimed is:

1. A quality control method for assessing products used for human in vitro fertilization; or Assisted Reproductive Technologies (ART) comprising:
providing a transgenic embryo (at least one-cell);
culturing said embryo in vitro for a specified duration;
evaluating embryo development from one-cell or two-cell to blastocyst stage and beyond depending on the type of the assay; and
determining acceptability/failure of tested items based upon said qualitative and quantitative analyses of embryo development.

2. The method of claim 1, wherein said one-cell embryo comprises at least one transgene operably linked to at least one embryonic development/pluripotency marker.

3. The method of claim 2, wherein said transgene includes a reporter gene encoding a selected fluorescent protein, including but not limited to green fluorescent protein (GFP), red fluorescent protein, Cyan Florescent protein, Orange Fluorescent protein and yellow fluorescent protein.

4. The method of claim 3, wherein said test items are selected from the group consisting of gamete and embryo culture media, gamete and embryo handling/processing media (to include washing and separation media), transport media, enzymes for denuding oocytes, gradient for sperm separation, freezing/vitrification media, thawing/warming media, pipette and embryo handling devices, lab-ware used in the process of human in vitro fertilization including but not limited to Petri dishes, centrifuge tubes, cryopreservation and Cryo-storage devices, and any solutions, reagents or devices involved with in vitro ART related procedures.

5. The method of claim 1, wherein said embryo evaluation is accomplished by assessing embryo morphology related to the developmental stages of the said embryos and the location/quantity/quality of fluorescence.

6. The method of claim 2, wherein said embryo is derived from mammalians including murine, porcine, equine, bovine, ovine and non-human primate

7. The method of claim 2, wherein said operably linked embryonic pluripotency markers including but not limited to Oct3/4, Sox2, Nanog as well as their upstream mediators and downstream effectors that play a role in ensuring normal embryo development.

8. The method of claim 1, further comprising evaluating embryo development at all stages including 1 and 2-cell-stages, 4-cell stage, 8-cells stage, morula stage, blastocyst stage and gastrulation stages.

9. A quality control assay for use in clinical ART to evaluate products used in the process of handling and preserving human gametes and producing, culturing and preserving human embryos comprising:
a transgenic one-cell embryo harvested from a transgenic mammal, wherein said embryo comprises at least one reporter gene operably linked to at least one embryonic pluripotency marker; and
instructions for evaluating ART products and IVF culture conditions, comprising:
incubating a transgenic one-cell embryo; and
evaluating embryo development based upon morphology from the one and two-cell to later blastulation and gastrulation stages.

10. The assay of claim 9, wherein said reporter gene encodes a protein selected from the group consisting of fluorescent proteins including but not limited to Green Fluorescent Protein, Cyan Fluorescent Protein, Orange Fluorescent Protein, Yellow Fluorescent Protein

11. The assay of claim 9, wherein said embryonic pluripotency markers, their upstream mediators and downstream effectors that play a role in ensuring normal embryo development.

12. The assay of claim 9, wherein said test items/growth conditions are evaluated based on embryo growth, development and quality based upon assessment of embryo morphology and qualitative/quantitative assessment of fluorescence. The acceptable threshold for optimal embryo growth is based on individual set criteria depending on test items.

13. The assay of claim 9, wherein said test items/growth conditions are embryotoxic.

14. An enhanced embryo assay (EA) for use in quality control of clinical human ART/IVF, comprising:
a transgenic one-cell embryo, said embryo comprising at least one reporter gene operably linked to at least one gene associated with embryonic development; and
an embryo expressing a transgenic/reporter gene differentially under optimal and sub-optimal or embryo-toxic culture conditions; and
an ART consumable.

15. The assay of claim 14, wherein said ART consumable is selected from the embryo culture media, gamete handling media, enzymes for denuding oocytes, gradient for sperm separation, freezing media, thawing media, pipettes and embryo handling devices, lab-ware used in the process of human in vitro fertilization including but not limited to Petri dishes, centrifuge tubes, cryopreservation and cryostorage devices.

16. A method for enhancing the sensitivity of embryo assay using embryo development to the blastocyst stage comprising:
providing a transgenic embryo comprising at least one reporter gene operably linked to at least one embryonic pluripotency marker;
incubating said transgenic embryo under culture conditions/test items; and
evaluating embryo development morphologically and via the expression of said embryonic marker from one-cell to blastocyst and gastrulation stages.

17. The method of claim 16, further comprising evaluating expression of said embryonic marker at the blastocyst stage and beyond (gastrulation).

18. The method of claim 16, wherein said evaluation comprises determining fluorescence of said reporter gene.

19. The method of claim 16, wherein said assay detects embryo-toxicity in culture media and/or culture materials.

20. The method of claim 16, wherein said assay detects functionality of media and suitability of materials used in clinical in vitro fertilization environments.

21. A modified, transgenic embryo, comprising at least one transgene operably linked to at least one embryonic pluripotency marker.

22. The embryo of claim 21, wherein said embryonic pluripotency markers and their upstream mediators and downstream effectors that play a role in ensuring normal embryo development.

23. The embryo of claim 21, wherein said transgene is a reporter gene.

24. The embryo of claim 23, wherein said reporter gene is a fluorescent or luminescent protein selected from the group consisting of green fluorescent protein, red fluorescent protein, cyan fluorescent protein, orange fluorescent protein, yellow fluorescent protein.
